(19) United States
(12) Patent Application Publication
(10) Pub. No.: US 2010/0192669 A1
(43) Pub. Date: Aug. 5, 2010

(54) PHOTO ACOUSTIC SAMPLE DETECTOR WITH LIGHT GUIDE

(75) Inventors: Cristian Presura, Eindhoven (NL); Hans Willem Van Kesteren, Eindhoven (NL); Michel Cornelis Josephus Marie Vissenberg, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., EINDHOVEN (NL)

(21) Appl. No.: 12/667,745

(22) PCT Filed: Jun. 30, 2008
(86) PCT No.: PCT/IB08/52627

§ 371 Date: Jan. 5, 2010

(30) Foreign Application Priority Data
Jul. 6, 2007 (EP) 07111904.4
Oct. 5, 2007 (EP) 07117960.0

(51) Int. Cl. G01N 33/497; G01N 21/00

(52) U.S. Cl. 73/23.3; 73/24.02

(57) ABSTRACT

A photo acoustic sample detector (10) is provided for detecting a concentration of sample molecules in a sample mixture (1). The photo acoustic sample detector (10) comprises an input for receiving the sample mixture (1), an acoustic cavity (3) for containing the sample mixture (1), a light source (5) for sending light (50) into the acoustic cavity (3) for exciting the sample molecules and thereby causing sound waves in the acoustic cavity (3) and a pick up element (4) for converting the sound waves into electrical signals (12). The photo acoustic sample detector (10) also comprises a light guide (2) comprising a transparent inner wall (8) at an interface of the light guide (2) and the acoustic cavity (3) and a reflective outer wall (7) at an outside of the light guide (2). The light source (5) is arranged for illuminating the light guide (2). The light guide (2) serves for reflecting the light (50) back and forth through the light guide (2) and the acoustic cavity (3).

### FIELD OF THE INVENTION

This invention relates to a photo acoustic sample detector for detecting a concentration of sample molecules in a sample mixture, the photo acoustic sample detector comprising, an input for receiving the sample mixture, an acoustic cavity for containing the sample mixture, a light source for sending light into the acoustic cavity for exciting the sample molecules and thereby causing sound waves in the acoustic cavity and a pick up element for converting the sound waves into electrical signals.

The invention further relates to a breath analysis device comprising such a photo acoustic sample detector.

### BACKGROUND OF THE INVENTION

Photo acoustic spectroscopy is a well known technique for measuring concentrations of different molecules in gases, down to ppb (parts per billion) level. This makes it suitable for measuring different molecules present in human breath. Generally lasers are used as light sources in photo acoustic spectroscopy. The laser light is collimated and the laser wavelength is tuned to excite the sample molecules into a higher energy level. This excitation leads to an increase of the thermal energy, resulting in a local rise of the temperature and the pressure inside the acoustic cavity. If the laser intensity is modulated at a resonance frequency of the acoustic cell, the pressure variations result in a standing acoustic wave. The acoustic waves are detected by a pick up element.

A disadvantage of the known photo acoustic sample detectors is that the optical alignment becomes very critical when the diameter of the acoustic resonator is small in order to obtain a low detection limit.

### SUMMARY OF THE INVENTION

It is an object of the invention to provide a photo acoustic sample detector wherein the optical alignment of the system is less critical. According to a first aspect of the invention, this object is achieved by providing a photo acoustic sample detector according to the opening paragraph, further comprising a light guide, the light source being arranged for illuminating the light guide, the light guide comprising a transparent inner wall at an interface of the light guide and the acoustic cavity and a reflective outer wall at an outside of the light guide for reflecting the light back and forth through the light guide and the acoustic cavity.

When the light arrives at the interface between the light guide and the acoustic cavity, it will pass the transparent wall, travel through the acoustic cavity and enter the light guide again at the light guide-cavity interface at the other side of the acoustic cavity. At an outer wall of the light guide, the light will be reflected. The reflected light may return to the acoustic cavity directly or via one or more additional reflections within the light guide. Because the light reflects back and forth through the light guide and the acoustic cavity, it passes the acoustic cavity many times. Each time the light passes the acoustic cavity it has a chance of exciting sample molecules. When the light passes the acoustic cavity more often, the sensitivity of the detector is significantly enhanced.

In the configuration according to the invention, the direction of the optical rays is not as critical as in the prior art, which enables the use of a divergent light source instead of the collimated laser beam of the prior art photo acoustic sample detectors. It is to be noted that it is known to use a combination of a collimated laser beam and a multi pass configuration. However, that combination needs a highly accurate optical alignment. Furthermore, to enable the multiple beams to pass the acoustic resonator its diameter has to be increased which makes the detection limit worse. According to the invention, the use of the light guide with a transparent light guide-cavity interface and reflective outer walls obviates those strict alignment requirements of the prior art and allows the use of a small diameter acoustic resonator improving the detection limit.

The photo acoustic sample detector according to the invention may use a collimated or uncollimated diode laser as light source, but preferably, the light source comprises at least one light emitting diode (LED). The LED should have an emission spectrum, overlapping an absorption spectrum of the sample molecules. The diode lasers used for known photo acoustic sample detectors have a temperature dependent wavelength. When the laser is not temperature stabilized, the measurement is susceptible to temperature variations. For, e.g., NO₂ concentration detection a blue LED forms a very attractive light source because the NO₂ absorption spectrum is very broad (so all the LED light is available for sensing) while the broader spectrum of the LED compared to the fine structure in the NO₂ absorption spectrum lead to an averaged spectral response which is relatively insensitive to the central wavelength and temperature of the LED. Moreover, LEDs are usually cheaper than diode lasers and available with higher output powers. The prior art photo acoustic sample detectors do generally not use LED light, because it can not be easily collimated and sent along the tube of the acoustical cavity.

The light guide is preferably made of a material with a low optical absorption to prevent photo acoustic signal generation in the light guide which could lead to a background photo acoustic signal during photo acoustic detection of the sample. The outer reflecting walls of the light guide can be made of metal or use can be made of total internal reflection at the light guide walls. When a metal is used, a small part of the light will be absorbed during reflection leading to a photo thermal response. However, due to the fact that the optical light guide thermally isolates the metal reflectors from the acoustic cavity, this will introduce no photo acoustic background signal.

An embodiment of the photo acoustic sample detector according to the invention further comprises an additional light emitting diode with an emission spectrum that is mainly outside the absorption spectrum of the sample molecules. Because the light from the additional LED does not contribute to the detector signal by exciting sample molecules, this additional LED can be used to compensate for background signals caused by light absorption in the neighborhood of the light guide-acoustic cavity interface.

Preferably, the pick up element is optically shielded from the light from the light guide in order to reduce direct excitation of the pick up element.

In one embodiment, a cross section of the light guide is arranged to provide a spiral light path for guiding the light spirally through the light guide and the acoustic cavity from an outer radius of the light guide to an inner radius, such that the light passes through the acoustic cavity twice per rotation. In this embodiment, the number of times that the light passes the cavity is optimized by guiding the light to the acoustic cavity and preventing light from bouncing back and forth in the light guide without passing the acoustic cavity at all. These and other aspects of the invention are apparent from and will be elucidated with reference to the embodiments described hereinafter.

### BRIEF DESCRIPTION OF THE DRAWINGS

In the drawings:

FIG. 1 shows an exemplary photo acoustic sample detector according to the invention,

FIG. 2 shows another photo acoustic sample detector,

FIG. 3 shows a cross section of a photo acoustic sample detector with a light guide with a circular shape,

FIG. 4 shows a cross section of a photo acoustic sample detector with a spiral light guiding pattern, and

FIG. 5a shows a cross section of a photo acoustic sample detector with two sample volumes and FIG. 5b shows the accompanying sample flow path.

### DETAILED DESCRIPTION OF THE INVENTION

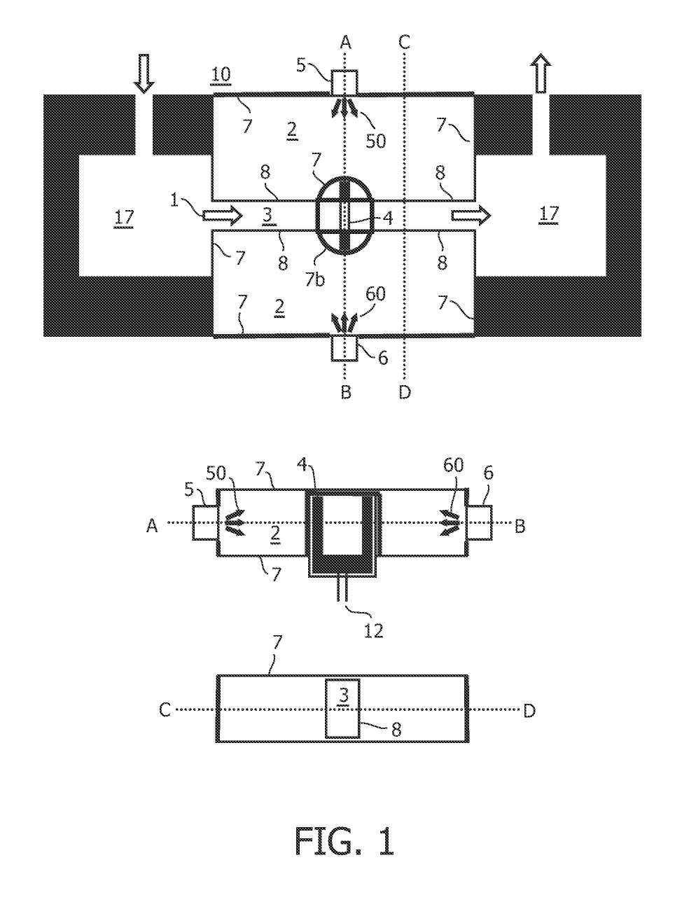

FIG. 1 shows three different cross sections of a photo acoustic sample detector 10 according to the invention. The sample detector 10 comprises an acoustic cavity 3 for receiving and containing a gas mixture 1. The buffer volumes 17 also contain the gas mixture. The cavity and buffer volumes may be closed for holding the gas mixture 1 or form a flow channel through which the gas mixture 1 may flow, e.g., during an exhalation of a user. It is to be noted that photo acoustic sample detection is also used for detecting sample concentrations in other sample mixtures, such as liquids or solids. In the following, the sample mixture is a gas mixture 1, but a skilled person would certainly be able to adapt the teachings of this document to other sample mixtures. In the embodiments discussed below, the acoustic cavity 3 has an elongated tubular shape, but in other embodiments, other shapes may be used for the acoustic cavity 3. A pick up element 4 in the acoustic cavity 3 serves for registering sound waves caused by variations in the thermal energy of the sample molecules. The thermal energy of the molecules is influenced by light 50 from the light source 5. Modulation of the light 50 from the light source 5 results in the variation of the thermal energy of the sample molecules. The light source 5 may, e.g., be a light emitting diode (LED) or diode laser. The sample detector 10 may comprise multiple sources 5 at various positions. It is important that the wavelength spectrum of the light 50 from the light source 5 comprises (a) wavelength(s) in the absorption spectrum of the sample molecules, in order to be able to excite the sample molecules and produce sound waves. The light modulation may be performed using wavelength modulation (mainly for laser diodes) or using intensity modulation (for laser diodes and LEDs). Preferably, to allow standing waves to occur, the length of acoustic resonator 3 is chosen to correspond to half the acoustic wavelength generated by amplitude modulation or frequency modulation of the light source. The lengths of the buffer volumes are a quarter of the acoustic wavelength. The pick up element 4 may be a microphone or other type of transducer for converting audio waves to a usable (electric) signal 12. Preferably a tuning fork element, e.g. a quartz crystal tuning fork is used as a pick up element 4.

The light 50 is guided to the acoustic cavity 3 by a light guide 2. The light guide 2 is made of glass, quartz, PMMA or another (mixture of) material with a low absorption at the wavelengths applied. Preferably, all walls 8 at the interface of the light guide 2 and the acoustic cavity 3 are transparent for allowing the light 50 to enter or leave the cavity 3. All other walls 7 are preferably reflective, for reflecting a high percentage of the light 50 back into the light guide 2. For reflecting the light, total internal reflection, a metal reflection layer or an appropriately chosen dielectric layer stack can be used. At the walls where the rays have an angle suitable for total internal reflection, this method is preferred because the percentage of the light reflected is higher than for reflection on a metal or dielectric layer stack. While the light 50 reflects back and forth within the light guide, it may cross the acoustic cavity 3 many times. The average number of times that the light 50 crosses the acoustic cavity 3 before it is absorbed in the material of the light guide 2 or leaves the light guide 2 at an outer wall 7, may be increased by coating the transparent walls 8 with an antireflective coating. In the embodiment shown in FIG. 1 a planar light guide is used. In that case it is attractive to use a rectangular cross section of the acoustic resonator. Preferably, the pick up element 4 is shielded from the light 50 coming from the light guide 2 to avoid the pick up element 4 being excited by the light 50 instead of by the sound waves. This is especially relevant when the light source is amplitude modulated and the pickup element is a tuning fork. The shielding may be realized using a reflective coating 7 at the inner side of the wall of the light guide 2. The cavity side of this wall should not be reflective, because that would direct light to the pick up element 4 and thereby increase background signal. The pick up element 4 may also be thermally shielded from the light guide 2 to avoid that light absorption at the light guide-cavity interface near the pick up element 4 has a direct thermal effect on the pick up element. Electrical connections 12 are provide to the pickup for signal detection.

An additional light source 6 may be provided for enabling background signal compensation. The additional light source 6 emits light 60 at a wavelength that is not or significantly less absorbed by the sample molecules. Consequently, detector signals caused by this additional light source 6 will mainly be caused by direct excitation of the pick up element 4 by the light 60 or by a thermal effect of light 60 that is absorbed at the cavity-light guide interface. The background signal originating from the additional light source 6 may be used for compensating the measurements performed with the main light source 5. Preferably, both light sources 5 and 6 are modulated in antiphase. The intensity of the light source 6 is chosen such that the background signals from both sources cancel each other. The modulated light intensity of the main light source 5 will cause sound waves with an amplitude depending on the sample concentration.

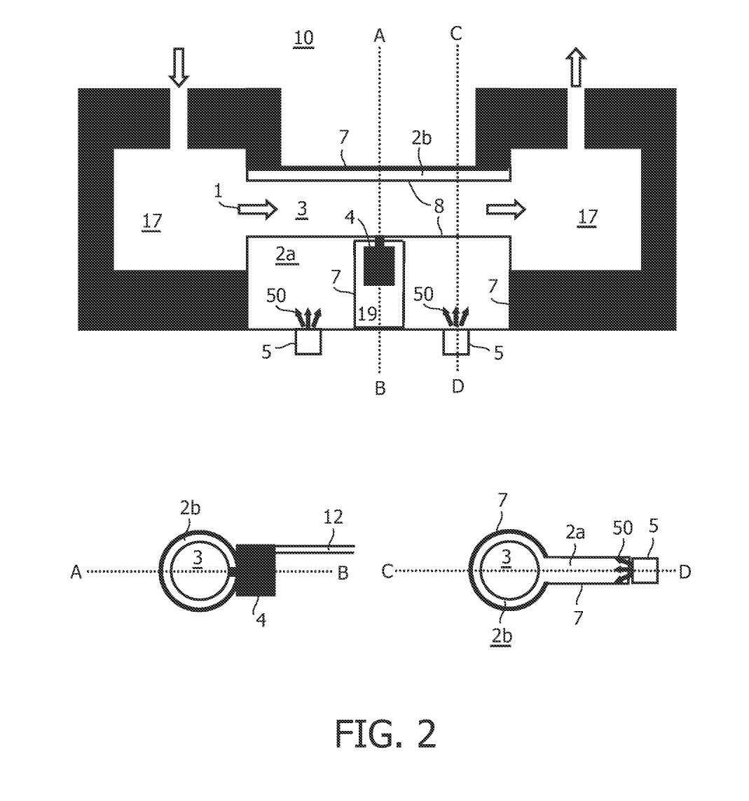

FIG. 2 shows a photo acoustic sample detector 10 with a light guide and an acoustic resonator having a circular cross section. The light guide is split in two parts having separate light sources 5. A first planar part 2a spreads the light along the longitudinal direction of the acoustic resonator. A second circular part 2b is used for reflecting the light multiple times through the acoustic cavity. No light passes in the section 19 in between the light guides 2a to prevent direct excitation of the pickup element 4. Here, the pickup element 4, for instance a microphone is placed outside the acoustic resonator. A small hole in the acoustic resonator couples the sound waves to the pickup element 4. It is an advantage of the light guide shown in FIG. 2, that more light 50 reflecting back from the outer walls 7 of the light guide 2b is directed towards the acoustic cavity 3. In, e.g., the configuration of FIG. 1, light 50 may reflect between two walls 7 of the light guide 2, without ever passing the cavity-light guide interface. When the difference between the outer and inner diameter of the light guide 2b in the configuration of FIG. 2 is small most of the light 50 leaving the acoustic cavity 3 will only need one reflection to return to the acoustic cavity 3. Because, in the end, all light that is not absorbed by the sample molecules will be absorbed somewhere in the detector 10 or will somehow escape the detector 10, it is important that the light passes the acoustic cavity 3 as often as possible and that the amount of non-effective internal reflections within the light guide 2 is minimized.

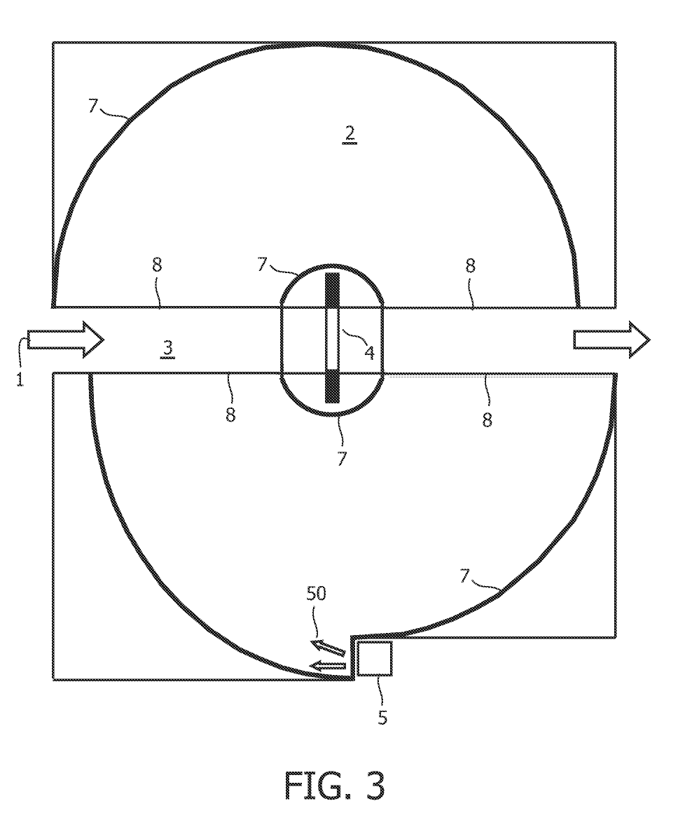

FIG. 3 shows a cross section of a photo acoustic sample detector with a planar light guide like the embodiment shown in FIG. 1 but having a circular cross section in the plane of the acoustic resonator.

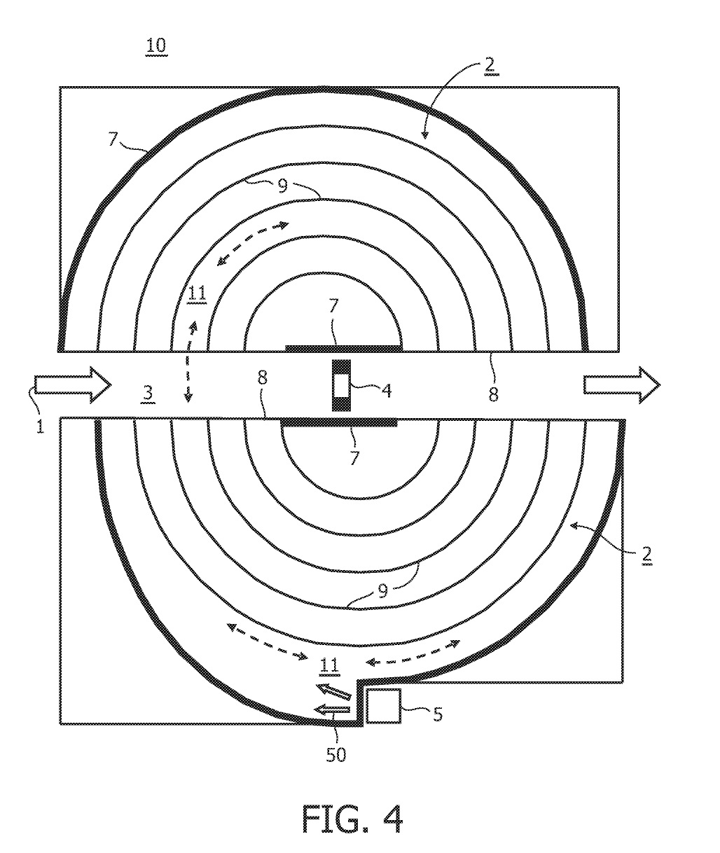

FIG. 4 shows a cross section of a photo acoustic sample detector 10 with a planar spiral light guiding pattern 11. In this configuration, the semi-circular parts of the light guide 2 are constructed of multiple semi-circular paths, leading the light 50 from the outer side of the acoustic cavity to the inner side. The light paths 11 are provided by adding internal reflective walls 9 to the semi-circular light guide parts, already shown in FIG. 3. The internal reflective walls 9 bend or reflect the light in such a way that it follows the light path 11. The reflective walls may, e.g., comprise a reflective coating or may be made of a material with a refractive index, different from the other parts of the light guide. Alternatively, the reflectivity of the internal walls 9 is caused by total internal reflection by providing air gaps between the paths 11. In the embodiment of FIG. 4, the number of times that the light passes the acoustic cavity 3 is optimized by guiding the light to the acoustic cavity 3 and preventing light from reflecting back and forth in the light guide 2 without passing the acoustic cavity 3 at all.

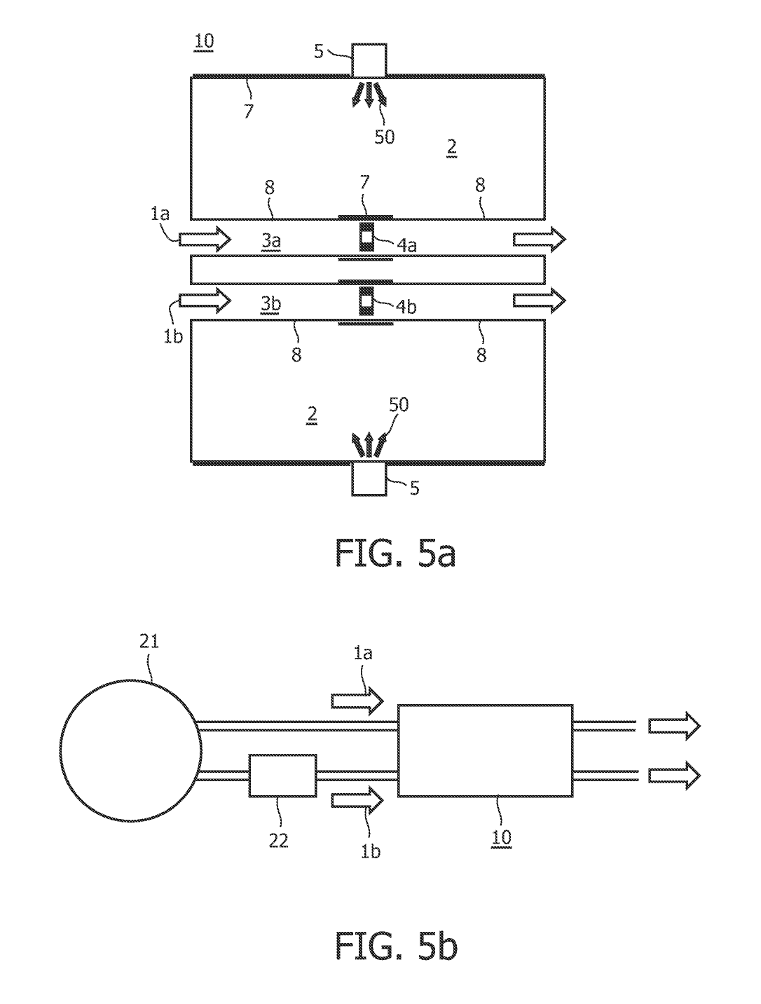

FIG. 5a shows a configuration with a light guide encompassing two acoustic cavities 3a, 3b and two sample flows 1a, 1b. The light 50 from the sources 5 passes through both acoustic resonators. The tuning fork pickup elements 4a, 4b are placed inside the acoustic resonators. The tuning forks are connected in a differential mode and the molecules to be sensed are only present in one of the two sample streams. In this way background signals from a number of origins can be cancelled simultaneously without the use of two wavelength light sources as in the embodiment of FIG. 1. This embodiment can, e.g., be used advantageously in combination with the technology described in patent WO 2006/114766 for the detection of nitrogen-containing compounds in a gas mixture. An example forms the detection of NO in the exhaled breath. As indicated in FIG. 5b, part of the exhaled breath 21 passes through flow channel 1a. Another part passes converter 22 which converts NO into NO₂ before it enters flow channel 1b. The sensor applies a blue LED for the photo acoustic detection of NO₂. During the exhalation the CO₂ and O₂ concentrations will vary in both flow channels in the same way. Interfering effects on the photo acoustic NO detection by these varying CO₂ and O₂ concentrations will be cancelled during the differential detection. Background signals generated in the sensor for instance by light absorption in the light guide around the acoustic resonators are also cancelled in this scheme.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A photo acoustic sample detector (10) for detecting a concentration of sample molecules in a sample mixture (1), the photo acoustic sample detector (10) comprising,
an input for receiving the sample mixture (1),
an acoustic cavity (3) for containing the sample mixture (1),
a light source (5) for sending light (50) into the acoustic cavity (3) for exciting the sample molecules and thereby causing sound waves in the acoustic cavity (3),
a pick up element (4) for converting the sound waves into electrical signals (12), and
a light guide (2),
the light source (5) being arranged for illuminating the light guide (2), the light guide (2) comprising a transparent inner wall (8) at an interface of the light guide (2) and the acoustic cavity (3) and a reflective outer wall (7) at an outside of the light guide (2) for reflecting the light (50) back and forth through the light guide (2) and the acoustic cavity (3).

2. A photo acoustic sample detector (10) as claimed in claim 1, wherein the light source (5) comprises at least one light emitting diode with an emission spectrum, overlapping an absorption spectrum of the sample molecules.

3. A photo acoustic sample detector (10) as claimed in claim 2, further comprising an additional light emitting diode (6) with an emission spectrum that is mainly outside the absorption spectrum of the sample molecules.

4. A photo acoustic sample detector (10) as claimed in claim 3, further comprising a light modulator for providing an antiphase modulation of the intensities of the light emitting diode (5) and the additional light emitting diode (6).

5. A photo acoustic sample detector (10) as claimed in claim 1, wherein the light guide (2) comprises glass.

6. A photo acoustic sample detector (10) as claimed in claim 1, wherein the transparent inner wall (8) comprises an antireflective coating and the reflective outer wall (7) comprises a reflective coating.

7. A photo acoustic sample detector (10) as claimed in claim 1, wherein the pick up element (4) is a tuning fork.

8. A photo acoustic sample detector (10) as claimed in claim 1, wherein the pick up element (4) is optically shielded from the light from the light guide in order to reduce direct excitation of the pick up element (4).

9. A photo acoustic sample detector (10) as claimed in claim 1, wherein the pick up element (4) is thermally shielded from the light guide (2).

10. A photo acoustic sample detector (10) as claimed in claim 1, wherein the acoustic cavity (3) has an elongated shape and wherein the light guide (2) is arranged such that the light (50) passes the acoustic cavity (3) transversally.

11. A photo acoustic sample detector (10) as claimed in claim 1, wherein a cross section of the light guide (2) comprises two semicircles, situated at opposite sides of the acoustic cavity (3).

12. A photo acoustic sample detector (10) as claimed in claim 11, wherein a cross section of the light guide (2) is arranged to provide a spiral light path (11) for guiding the light (50) spirally through the light guide (2) and the acoustic cavity (3) from an outer radius of the light guide (2) to an inner radius.

13. A photo acoustic sample detector (10) as claimed in claim 12, wherein the spiral light path (11) is provided by reflective walls (9) inside the light guide (2).

14. A photo acoustic sample detector (10) as claimed in claim 12, wherein the spiral light path (11) is provided by air gaps inside the light guide (2).

15. A breath analysis device comprising a photo acoustic sample detector (10) as described in claim 1.
